(19) United States
(12) Patent Application Publication
(10) Pub. No.: US 2015/0265352 A1
(43) Pub. Date: Sep. 24, 2015

(54) ENTERAL NUTRITION DELIVERY SUPPORT SYSTEM

(71) Applicant: Patricia Johnson, Robbinsville, NJ (US)

(72) Inventor: Patricia Johnson, Robbinsville, NJ (US)

(21) Appl. No.: 14/731,840

(22) Filed: Jun. 5, 2015

Related U.S. Application Data
(63) Continuation of application No. 29/349,186, filed on Mar. 15, 2010, now abandoned.

(51) Int. Cl. A61B 19/02; A61J 15/00

(52) U.S. Cl. CPC A61B 19/0264; A61J 15/0003; A61J 15/0076; A61B 2019/0265; A61B 2019/0278; A61B 2019/0286; A61B 2019/0285; A61B 2019/0279

(57) ABSTRACT

An enteral nutrition delivery support system having two separate ultra lightweight soft-shell carrying cases. An enteral feeding pump is carried in one case. An IV liquid food bag is stored in the other case. The two carrying cases are fitted with portals which allow polyethylene food supply and delivery tubes to be connected to the pump and feed bag. The carrying cases are worn around the patient's waist with a reinforced nylon belt.

### CROSS-REFERENCE TO RELATED APPLICATIONS

This application is a continuation of U.S. Design patent application No. 29/349,186 filed on Mar. 15, 2010, which is herein incorporated by reference in its entirety.

### BACKGROUND OF THE INVENTIVE FIELD

The present invention is directed to an enteral nutrition delivery support system comprised of an ultra light-weight “tube feeding” delivery support system for patients who need transportable enteral nutrition in order to enjoy the “full range” of life's physical activities.

Patients unable to eat food, due to illness, decreased appetite, or some type of obstruction or surgery which interferes with swallowing, must be supplied nutrition in a different way. One method of providing patients sustenance is called enteral nutrition or “tube feeding.” This process provides the patient nutrition through a tube which is placed into the stomach or small bowel. The food itself is a special liquid mixture containing necessary proteins, carbohydrates (sugar), fats, vitamins and minerals.

Tube feeding impacts patients of all ages, infants as well as adults, who require various lengths of time for enteral feeding. Sometimes tube feeding is required for only a short period of time. These patients will have the tube removed once they are able to eat normally again. Other patients may requite enteral tube feeding for much longer, and in some cases, for the rest of their lives.

There are different types of feeding tubes. Nasogastric or nasoenteral tube feeding is accomplished through the nose or mouth. Gastrostomy or jejunostomy is when a tube is placed directly through the skin into the stomach or bowel.

Medical technology has advanced to the point where many patients are surviving longer. At the same time, new and challenging diagnoses, and the innovative treatments that doctors have employed to save lives, have the collateral consequence of increasing the number of patients who require enteral nutrition for much longer periods of time, and in some cases for the rest of their lives. Sadly, the number of children and adolescents diagnosed with gastrointestinal diseases is increasing. Some will require a lifetime of enteral tube feeding.

As a result of recent comparison studies, enteral nutrition is likely to be prescribed more frequently. Studies have found enteral nutrition to be a safer (less susceptible to life-threatening post-operative infections) and better way of providing patients sustenance than parenteral nutrition (the administration of nutrients by a route other than the alimentary canal, such as subcutaneously, intravenously, intramuscularly, or intradermally).

Delivering enteral nutrition is a slow and tedious process, requiring the patient to be tube fed for up to 12 hours a day, either continuously or divided into three feeding sessions of 4 hours each.

The typical patient's first experience with enteral nutrition occurs in a hospital setting. The patient's feeding tube is connected to an enteral feeding pump mounted to a hospital IV stand with a liquid IV food bag hanging above it on a hook. If a patient is discharged from the hospital, this same delivery support system, i.e., the feeding pump and feed bag mounted to a hospital IV stand, would be used.

The process is one that often relegates a patient to their bed for 24 hours a day. The long-term impact on the patient, both physically and psychologically, can be devastating, frustrating the patient's physical recovery and sending patients into depression—sometimes severe depression with suicidal ideation.

As a result of recent innovations, patients who need long-term enteral nutrition have been able to get out of their beds. Delivery support systems currently in use include backpacks and shoulder bags. These innovations can accommodate a battery powered enteral feeding pump and liquid food bag which are placed into the backpack or shoulder bag and transported by the patient. Both innovations enable the patient to ambulate while receiving enteral nutrition. However, these innovations have both technological and physical limitations—some of which were dangerous for the patient.

Access to the enteral feeding pump is important. First and foremost, the pump must be turned on and off when needed. Additionally, enteral feeding pumps have a panel of controls that require occasional adjustment by the patient. New feeding tubes and new liquid food delivery tubes must also be connected to and from the pump from time to time. The pump needs to be recharged, batteries have to be changed and, on occasion, the pump itself must be replaced. Ready access to the pump is important.

Backpacks and shoulder bags do not provide the patient ready access to the enteral feeding pump. Every time the patient needs to turn the pump on or off, make a pump adjustment, change a feeding tube, or replace batteries, the patient must remove the backpack or shoulder bag, set it down, open the bag, and either fumble around in the bag or remove the feeding pump in order to make the necessary pump adjustment or connection. The patient must then put the backpack or shoulder bag back on, being careful not to entangle or disconnect a tube, and readjust the backpack or shoulder bag for comfort.

These repeated movements carry associated risks. Every time the patient needs to remove or replace the backpack or shoulder bag, there is a risk that a feeding tube may become entangled, or worse, that a feeding tube may accidentally disconnect from the pump or the patient in an unsterile environment, creating a serious risk of infection and interrupting the feeding cycle. Further, every time a patient removes the feeding pump, there is a risk that the patient may drop and damage the pump, tear out the feeding tube, or tear off a hose bib.

The very process of removing the backpack or shoulder bag can also be tedious and cumbersome, so much so that a patient may neglect making a necessary pump adjustment to avoid the hassle, compromising both the efficiency of the pump and the adequacy of the nutrition being delivered to the patient.

The backpack models are also much larger than the enteral feeding machine. This increases risk to the patient because the machine and tubing connections are constantly bouncing around in the bag as the patient moves. Again, the tubing may become entangled or disconnect without the patient's knowledge. This also increases the risk that the power switch or some other panel adjustment on the pump may be accidentally triggered, shut off, or disabled.

The backpack and shoulder bag models are plagued by other problems related to changing liquid feed bags. Because the liquid feed bag is stored in the same enclosure as the enteral feeding pump (shoulder bag), or externally attached to the same backpack, changing liquid feed bags imposes the same set of obstacles and creates the same associated risks that patients encounter with gaining access to the enteral feeding pump. Not only must the patient remove the backpack or shoulder bag every time he or she wants to gain access to the pump, they must remove the backpack or shoulder bag every time they need to change liquid feed bags.

Finally, by enclosing a pliable IV liquid feed bag in the same enclosure as a hard and asymmetrical object like the enteral feeding pump (shoulder bag), there is the risk that a feeding bag may be damaged or punctured as the enteral feeding pump bounces around.

In addition to technical limitations, backpacks and shoulder bags pose a number of physical limitations which can also have adverse psychological effects and prevent patients from enjoying the “full range” of life's physical activities.

Backpacks and shoulder bags are both physically obvious to onlookers. Patients often draw unwanted attention and stares when people see plastic feeding tubes dangling from a backpack or shoulder bag. Many patients feel uncomfortable wearing backpacks and shoulder bags because they know even well-meaning people may stare or ask intrusive questions to satisfy their natural curiosity. Patients may be reluctant to wear backpacks and shoulder bags in public if it means sacrificing their right to medical privacy, or if they feel stigmatized by others.

Backpacks and shoulder bags are also ergonomically unfriendly. The feeding pump, IV feeding bags and tubing are carried around on the upper back and shoulders. The total weight of the device, together with the weight of the backpack or shoulder bag, can be between 5 and 10 pounds. Carrying the weight of the feeding machine in a backpack or shoulder bag for any length of time places undue strain on the shoulders and back which can effect posture and cause pain.

Moreover, while backpacks and shoulder bags are great for transporting objects from one place to another, they are not designed to be worn for physical activities like golf, basketball, baseball, or even jogging. The physical limitations are many and include everything from leaning back in a chair, to driving a car, to giving a grandchild a piggyback ride, or participating in a favorite sport or activity.

The enteral nutrition delivery support system of the present invention overcomes all of the major technological and physical obstacles associated with backpacks and shoulder bags. Patients can engage in the “full range” of life's physical activities, restoring their self-image and enabling them to live active and normal lives.

### SUMMARY OF THE GENERAL INVENTIVE CONCEPT

The enteral nutrition delivery support system preferably comprised of two separate ultra lightweight soft-shell carrying cases. An enteral feeding pump is carried in one case. An IV liquid food bag is stored in the other case. The two carrying cases are fitted with portals which allow polyethylene food supply and delivery tubes to be connected to the pump and feed bag. The carrying cases are worn around the patient's waist with a reinforced nylon belt. They are moveable and can be positioned on the patient's left side, right side, in front, in back, or anywhere on the belt depending on need or preference. The two cases are designed to carry everything the patient needs to provide portable enteral nutrition while participating in their favorite activity or sport. The case for the pump is designed with a viewing flap for accessing the machine controls, panel and display.

The foregoing and other features and advantages of the present invention will be apparent from the following more detailed description of the particular embodiments, as illustrated in the accompanying drawings.

### BRIEF DESCRIPTION OF THE DRAWINGS

The following detailed description of the example embodiments refers to the accompanying figures that form a part thereof. The detailed description provides explanations by way of exemplary embodiments. It is to be understood that other embodiments may be used having mechanical and electrical changes that incorporate the scope of the present invention without departing from the spirit of the invention.

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is a perspective view of one embodiment of the pump case of the present invention;

FIG. 2 is a front view of the case of FIG. 1 (with the front flap open);

FIG. 3 is a side view of the case of FIG. 2;

FIG. 4 is a top view of the case of FIG. 2;

FIG. 5 is a perspective view of one embodiment of the case that holds the feeding bag;

FIG. 6 is a front view of the case of FIG. 5 (showing top flap open);

FIG. 7 is a side view of the case of FIG. 6;

FIG. 8 is a top view of the case of FIG. 6;

FIG. 9 is a bottom view of the case of FIG. 6;

FIG. 10 is a back view of the case of FIG. 6;

FIG. 11 is a front view of the cases of the present invention with the carrying belt;

FIG. 12 is a front view of the case of FIG. 1 with the front flap open showing a machine pump installed;

FIG. 13 illustrates a side view of the pump case having tubes through the side opening;

FIG. 14 shows a bottom view of the case of FIG. 5 showing tubes through the bottom opening of the case;

FIG. 15 shows a front perspective view of both cases on the belt (top flaps in open position);

FIG. 16 shows a back perspective view of both cases on the belt (top flaps open);

FIG. 17 shows a bottom view of both cases lying flat on a surface; and

FIG. 18 shows a perspective view of both cases with a machine pump and feeding bag installed in the cases (front flap open).

### DETAILED DESCRIPTION OF EXEMPLARY EMBODIMENT(S)

The enteral nutrition delivery support system of the present invention is comprised of two ultra lightweight, soft-shell carrying cases. An enteral feeding pump is carried in one case (hereinafter the “pump case”). An IV liquid food bag (or feeding bag) is carried in the second case (hereinafter the “IV case”).

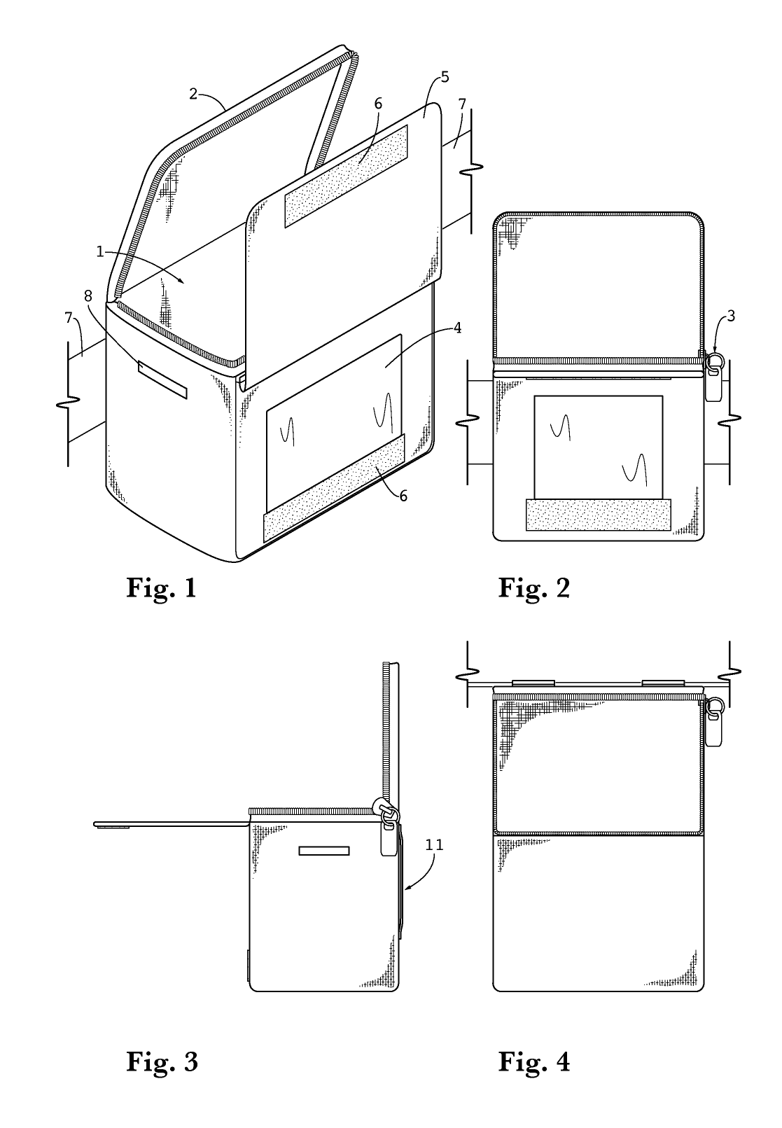

FIG. 1 is a perspective view of one embodiment of the pump case of the present invention. FIG. 1 is a perspective drawing which depicts the soft-shell case designed to carry an enteral feeding machine or pump.

In one embodiment of the invention, the pump case depicted in FIG. 1 is designed to carry the popular Kangaroo Joey enteral feeding pump. There are several manufacturers of enteral feeding pumps. These pumps come in a variety of sizes and shapes. The pump case of the present invention can be fabricated to accommodate any enteral feeding device. In one embodiment, the interior well of the pump case measures 4.1″×5.1″×3.6″. These dimensions are identical to the outer dimensions of the Kangaroo Joey enteral feeding pump. In the preferred embodiment, the enteral feeding pump fits snuggly into the well [1] of the pump case. Because of the snug fit, the machine pump does not have room to move around in the pump case.

The pump case has a top flap [2] which opens and closes on a fabric hinge as illustrated in FIG. 1. When closed, the top can be fastened with a heavy duty zipper[3] which runs along the top edge of the side and the front panels of the case.

The front panel of the pump case has a clear pliable polyethylene window [4] which is used to make touch screen pump control adjustments. The control window has a protective overhead front panel flap [5] which also opens and closes on a fabric hinge. The front flap can be closed over the control window and secured with a Velcro strip [6].

The right side panel and the left side panel of the pump case have portal openings [8] which allow pliable polyethylene food supply and food delivery tubes to pass through so they can be connected to the feeding pump. The exterior dimensions of the pump case are determined by the thickness of the fabric and soft-shell lining.

FIG. 2 is a front view of the case of FIG. 1 (with the front flap open-sticking straight out). FIG. 3 is a side view of the case of FIG. 2. FIG. 4 is a top view of the case of FIG. 2. As illustrated by the side view of the pump case depicted in FIG. 3, two vertical belt loops [11] are stitched to the back of the case. As further illustrated by FIG. 1, in one embodiment of the invention, a heavy duty nylon belt [7] passes through the belt loops. The nylon belt secures the pump case around the patient's waist. The pump case can then be moved along the belt and located anywhere on the patient's waist (front, back or sides).

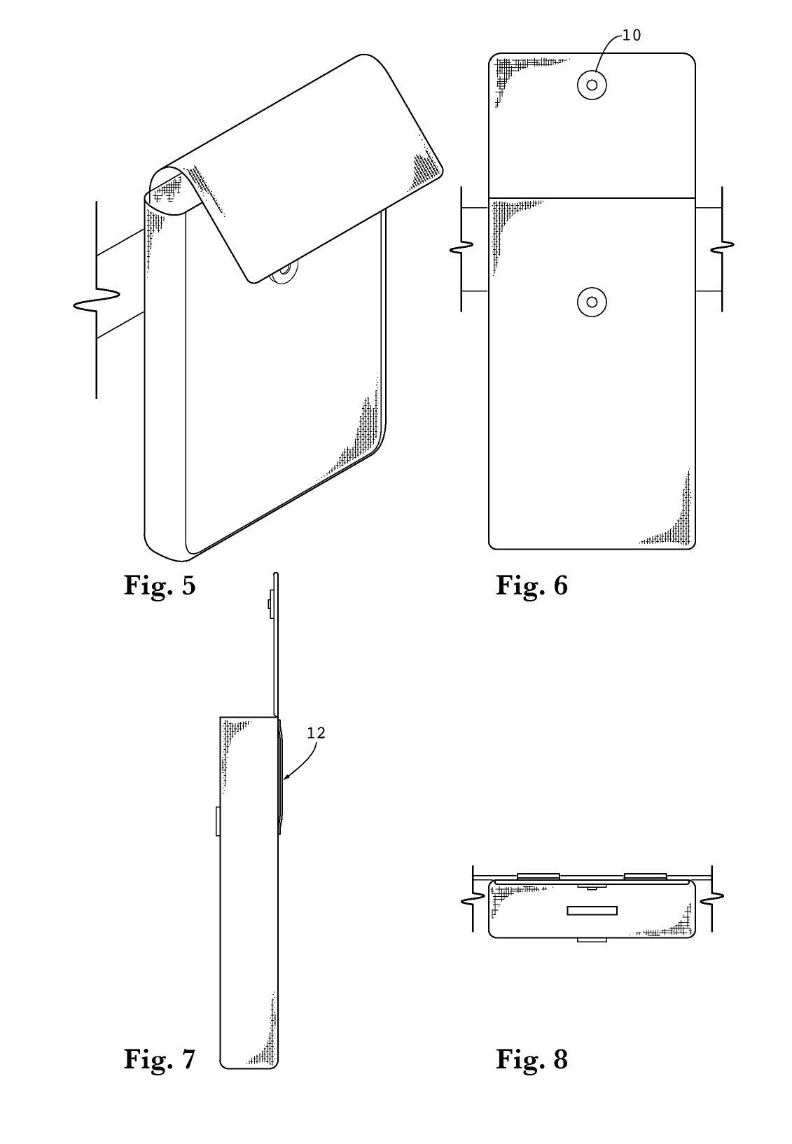

FIG. 5 is a perspective view of one embodiment of the case that holds the feeding case. In the preferred embodiment, the IV (or feeding) case is a soft-shell case designed to carry the IV liquid food bag. The IV case has a top flap which opens and closes on a fabric hinge as illustrated in FIG. 5. A magnetic button [10] secures the top flap to the front panel when closed.

The IV case depicted in FIG. 5 is designed to carry liquid bags of enteral nutrition. In one embodiment of the invention, the interior compartment of the IV case measures 8.5″×5″×1″. In this embodiment, the exterior dimensions of the IV case are determined by the thickness of the fabric and soft-shell lining.

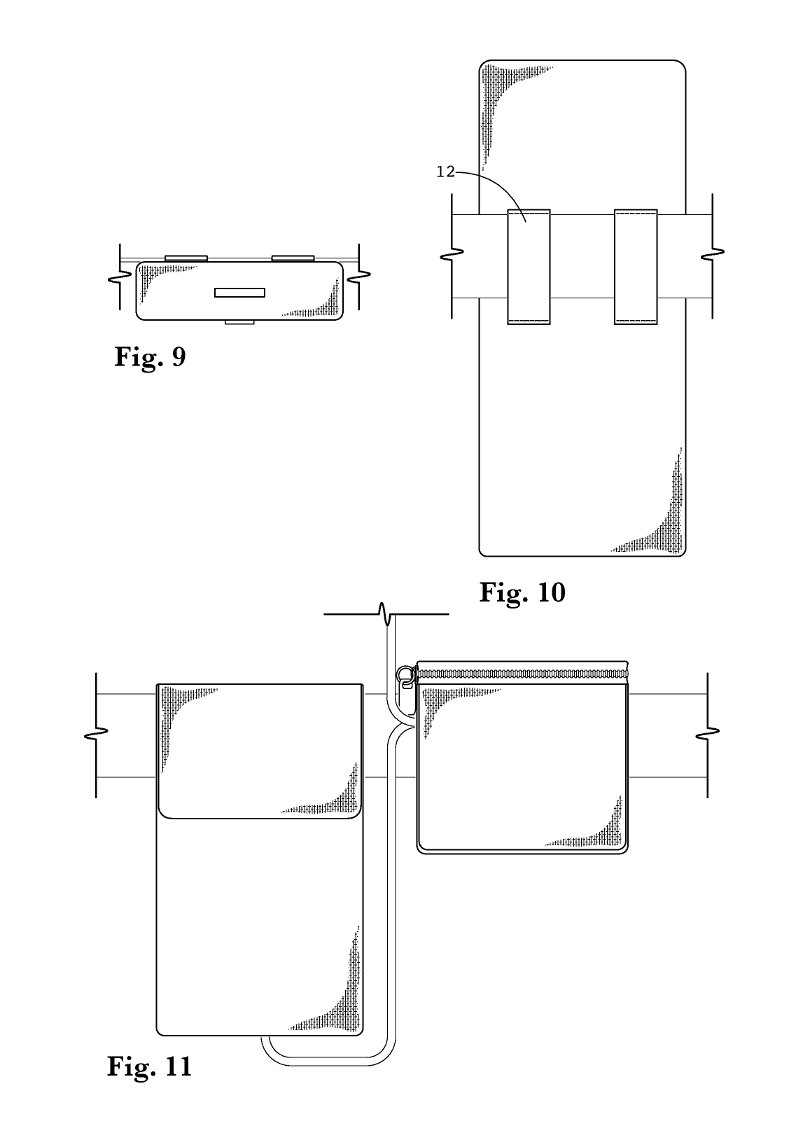

FIG. 6 is a front view of the IV case of FIG. 5 (showing top flap open). FIG. 7 is a side view of the case of FIG. 6. FIG. 8 is a top view of the case of FIG. 6. FIG. 9 is a bottom view of the case of FIG. 6. FIG. 10 is a back view of the case of FIG. 6.

As illustrated by FIG. 7, and as further illustrated by FIG. 10, two vertical belt loops [12] are stitched onto the back panel of the IV case. As further illustrated by FIG. 10, a heavy duty nylon belt passes through the belt loops. The nylon belt secures the IV case around the patient's waist. The IV case can be moved on the belt and can be positioned on either side of the pump case depending on medical need or patient preference.

As illustrated by FIG. 9, the bottom of the IV case has a portal opening which allows a pliable polyethylene food supply tube to pass through so it can be connected to the bag of liquid nutrition.

The pump case is constructed using three different materials. The exterior of the pump case, together with its top flap and front control window flap, is sewn together using a heavy-duty machine washable composite fabric. The interior of the pump case has a satin liner. Sandwiched between these two layers of fabric is a flexible waterproof polystyrene reinforcing insert. Together these materials form a durable soft-shell case which can withstand any weathering agent and protect the enteral feeding pump from moderate to severe impact.

As previously noted, a clear pliable polyethylene window is sewn into a rectangular opening in the front panel of the pump case which provides touch screen access to the control panel on the enteral feeding pump. The portal opening for the feed supply and delivery tubes are stitched into the side panels of the pump case. The zipper which secures the top flap to the side and front panels is a heavy duty model with a self-lubricating nylon track.

The IV case is constructed using the same materials and methods. Instead of using a zipper, however, the top flap is secured to the front panel using a magnetic button. The portal opening for the food supply tube is stitched into the bottom of the case.

When the machine pump is installed into the pump case of the present invention, a touch sensitive screen on the pump can be accessed from the front panel of the pump case. The user can lift the Velcro front panel flap and press the screen to make the desired adjustment. The pump case and IV case both provide convenient access for clearing or for connecting food supply and delivery tubes.

The IV case holds enough liquid food to take care of the patient's nutritional needs for an entire day. Should more nutrition be needed, replacing a feed bag can be accomplished without ever taking the belt off. The user can unsnap the top flap of the IV case, remove the old bag, disconnect the food delivery tube, connect the tube to the new bag, install, close the flap and resume the activity or sport.

FIG. 11 is a view of the cases of the present invention with the carrying belt. The carrying system of the present invention allows patients dependent on enteral feeding to enjoy active lives. The carrying case of the present system allows users to resume normal activities such as golf, fishing, sports, carpentry, and many other activities. By allowing patients to resume active, normal lifestyles, the system of the present invention provides emotional and physical benefits to the user.

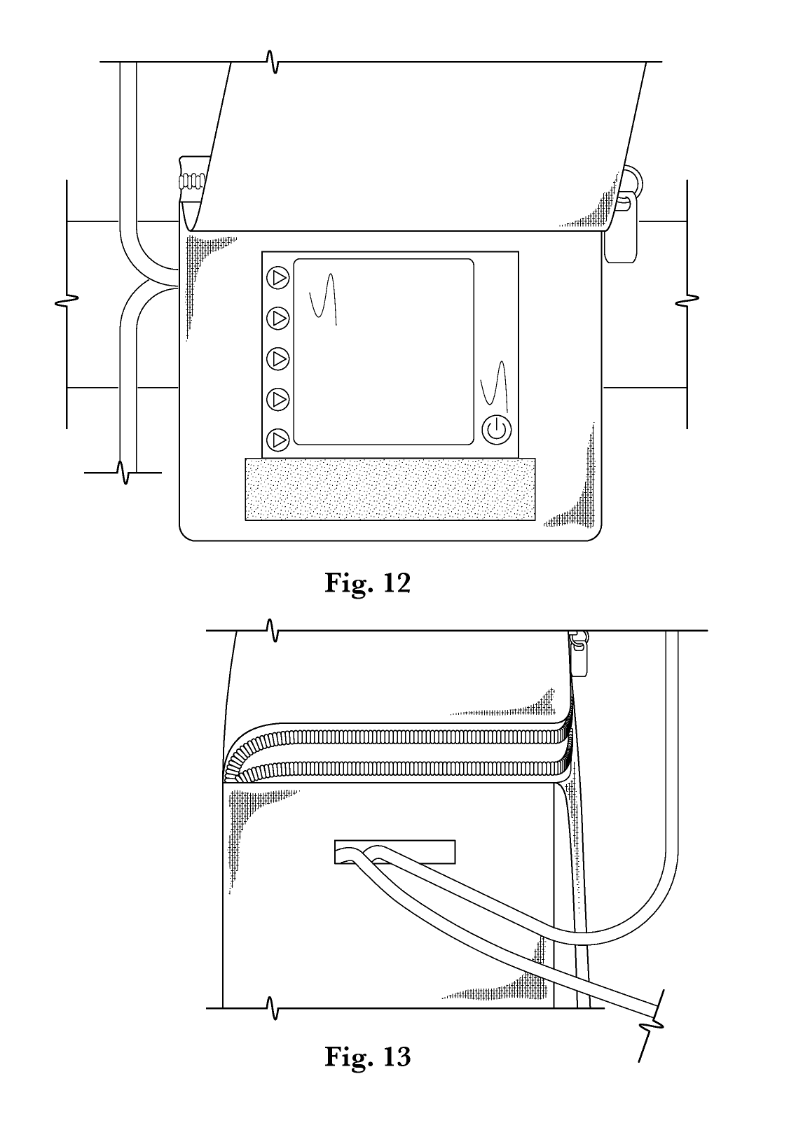

FIG. 12 is a front view of the case of FIG. 1 with the front flap open showing a machine pump installed. The enteral feeding pump and its panel of controls can be seen through a clear polyethylene touch sensitive control window. Polyethylene feeding tubes (both food supply and delivery tubes) can be seen entering and exiting a portal on the left side of the case. The portal provides an entry point to connect the food supply and delivery tubes to the pump. A similar portal on the right side of the case can be used to connect the food supply and delivery tubes if it is necessary or desirable to place the IV case on that side.

FIG. 13 illustrates a side view of the pump case having tubes through the side opening. This view provides a clear picture of the polyethylene feeding tubes (both food supply and delivery tubes) entering and exiting the portal which appears on the left side of the case.

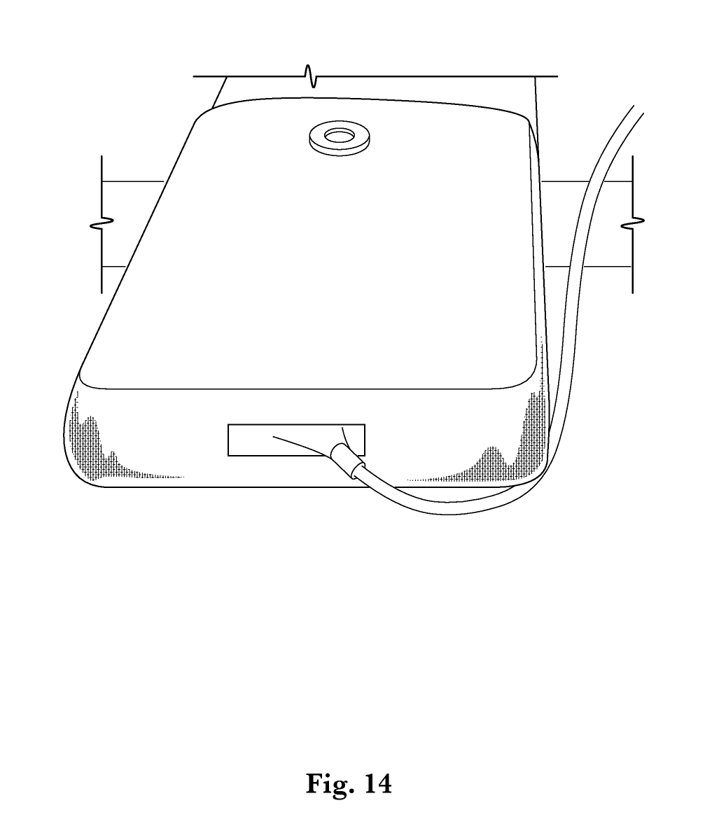

FIG. 14 shows a bottom view of the IV case of FIG. 5 showing tubes through the bottom opening of the case. This view provides a clear picture of the polyethylene food supply tube exiting the portal located on the bottom of the case. This tube connects to the machine pump as discussed above.

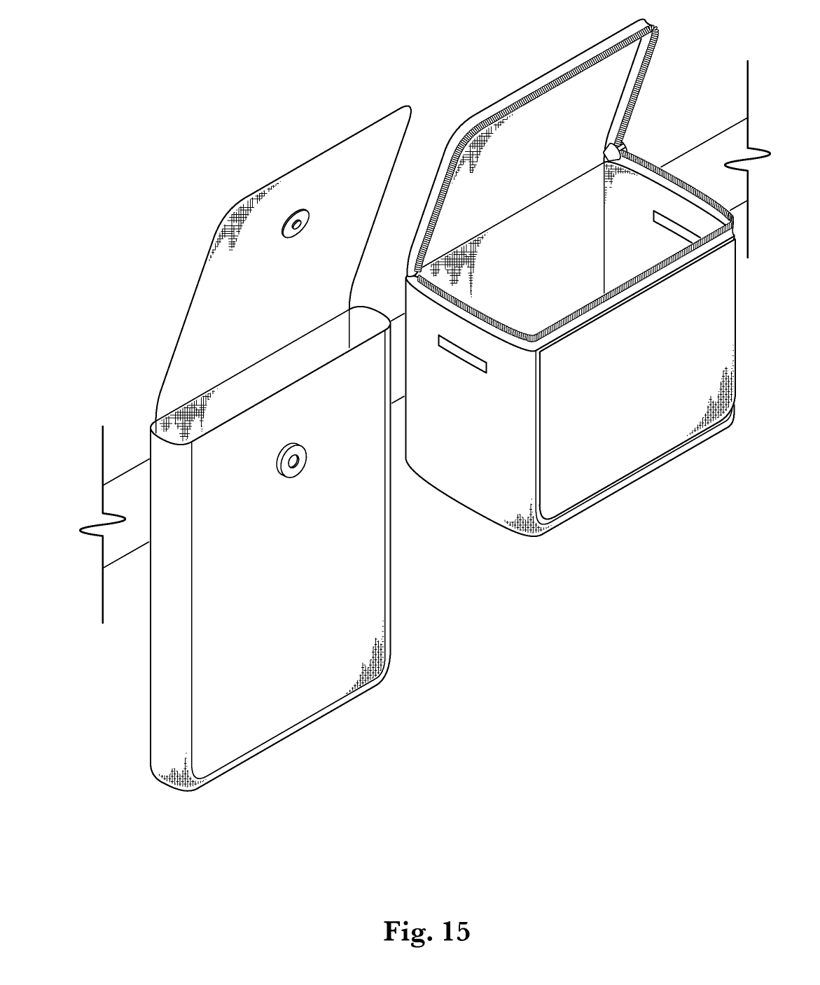

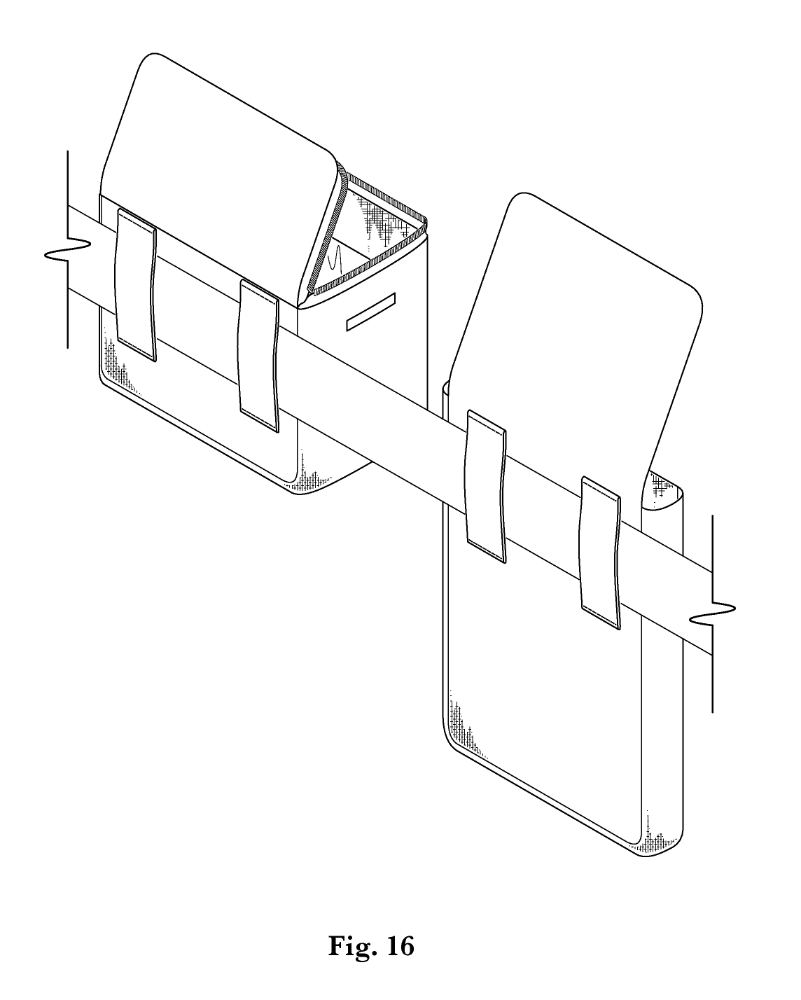

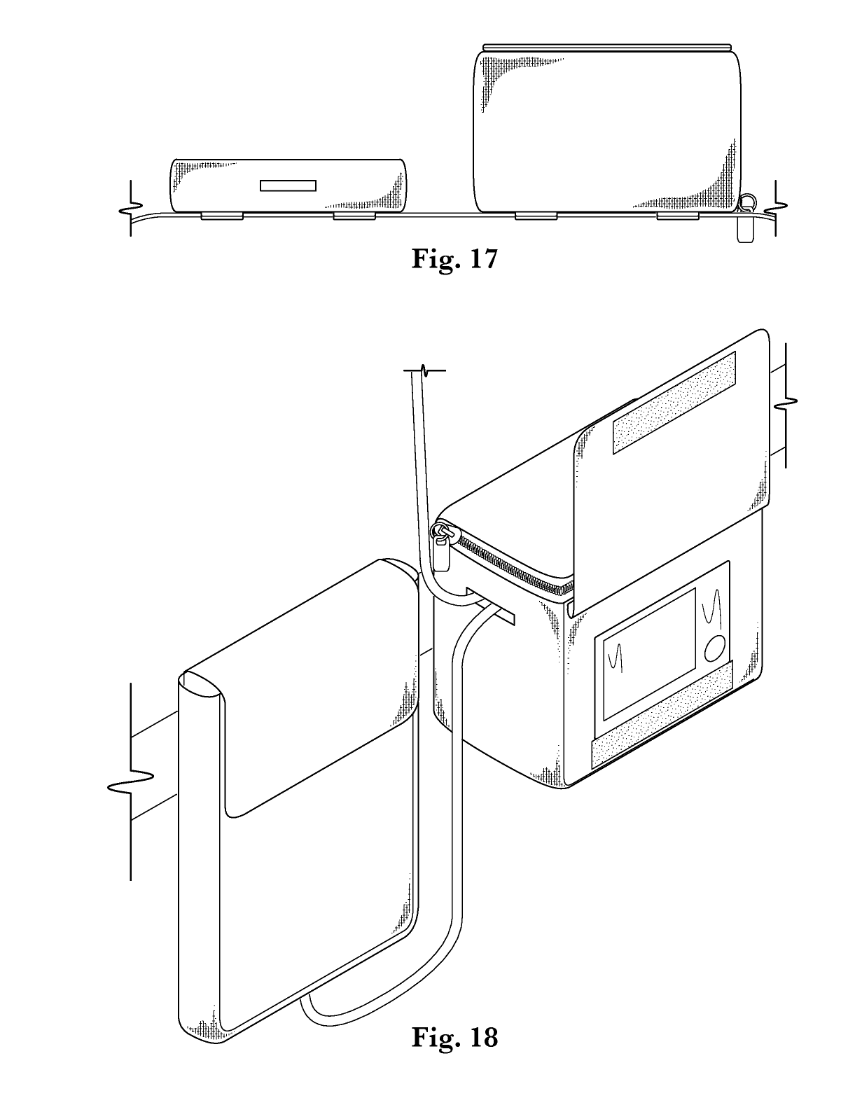

FIG. 15 shows a front perspective view of both cases on the belt (top flaps in open position). FIG. 16 shows a back perspective view of both cases on the belt (top flaps open). FIG. 17 shows a bottom view of both cases lying flat on a surface. FIG. 18 shows a perspective view of both cases with a machine pump and feeding bag installed in the cases (front flap open).

While certain embodiments of the present invention are described in detail above, the scope of the invention is not to be considered limited by such disclosure, and modifications are possible without departing from the spirit of the invention as evidenced by the following claims:

What is claimed is:

1. A system for providing nutrition delivery comprising:
a first case having an opening, the first case configured to accept a machine pump and configure to fit around the machine pump snuggly to prevent substantial movement of the machine pump while installed in the first case, the first case further comprised of:
a top flap for covering the opening in the first case;
a viewing window made of transparent material on the front of the first case for allowing touch access to controls on the machine pump;
a belt loop attached to the back of the first case;
a first portal on the first case for accepting tubes for connecting to the machine pump;
a second case having an opening, the second case configured to accept a feeding bag, the second case further comprised of;
a top flap for covering the opening in the second case;
a belt loop attached to the back of the second case;
a portal on the bottom of the second case for accepting a tube for connecting to the feeding bag;
a belt adapted for placement through the belt loops on the first and second cases.

2. A system according to claim 1, wherein the first portal on the first case is on a first side of the first case and further comprising, a second portal on a second side of the first case.

3. A system according to claim 2, wherein the user of the system can choose to place the second case on the first side of the first case or the second side of the first case when worn around a waist of the user.

4. A system according to claim 1, wherein the belt is adapted to be worn around the waist of a user and wherein the first and second cases can be positioned anywhere around the waist of the user and wherein the user can replace the feeding bag from the second case without removing the cases from the user's waist.

5. A system according to claim 1, wherein the first and second cases are made from soft-shell material.

6. A system according to claim 1, configured to allow a user to move and place the first and second cases anywhere around the user's waist.

7. A system according to claim 1, wherein the first and second case is comprised of an outer layer of heavy-duty machine washable material and an interior layer made of soft fabric.

8. A system according to claim 7, wherein the first and second case is comprised of a middle waterproof layer of material sandwiched between the outer and interior layers.

9. A system according to claim 1, wherein the portal on the bottom of the second case allows a tube from the feeding bag to be connected to the machine pump through the first portal on the first case.

10. A system according to claim 9, wherein the first portal on the first case allows a tube from the machine pump to deliver food to the user, and wherein the first case is further comprised of:
a front flap for covering the viewing window.

11. A system for providing nutrition delivery comprising:
a first case having an opening, the first case configured to accept a machine pump and configure to fit around the machine pump snuggly to prevent substantial movement of the machine pump while installed in the first case, the first case further comprised of:
a top flap for covering the opening in the first case;
a viewing window made of transparent material on the front of the first case for allowing touch access to controls on the machine pump;
a front flap for covering the viewing window;
a belt loop attached to the back of the first case;
a first portal on a first side of the first case for accepting tubes for connecting to the machine pump;
a second portal on a second side of the first case for accepting tubes for connecting to the machine pump;
a second case having an opening, the second case configured to accept a feeding bag, the second case further comprised of;
a top flap for covering the opening in the second case;
a belt loop attached to the back of the second case;
a portal on the bottom of the second case for accepting a tube for connecting to the feeding bag;
a belt adapted for placement through the belt loops on the first and second cases; and
wherein a user of the system can choose to place the second case on the first side of the first case or the second side of the first case when the first and second cases are placed on the belt around the user.

12. A system according to claim 11, wherein the belt is adapted to be worn around the waist of the user and wherein the first and second cases can be positioned anywhere around the waist of the user and wherein the user can replace the feeding bag from the second case without removing the cases from the user's waist.

13. A system according to claim 11, wherein the first and second cases are made from soft-shell material.

14. A system according to claim 11, configured to allow the user to move and place the first and second cases anywhere around the user's waist.

15. A system according to claim 11, wherein the first and second case is comprised of an outer layer of heavy-duty machine washable material and an interior layer made of soft fabric.

16. A system according to claim 15, wherein the first and second case is comprised of a middle waterproof layer of material sandwiched between the outer and interior layers.

17. A system according to claim 11, wherein the portal on the bottom of the second case allows a tube from the feeding bag to be connected to the machine pump through the first portal on the first case.

18. A system according to claim 17, wherein the first portal on the first case allows a tube from the machine pump to deliver food to the user.

19. A method for providing nutrition delivery to a user via a machine pump, having a control panel, and feeding bag comprising the steps of:
providing a first case having an opening, the first case configured to accept the machine pump and configure to fit around the machine pump snuggly to prevent substantial movement of the machine pump while installed in the first case, the first case further comprised of:
a top flap for covering the opening in the first case;
a viewing window made of transparent material on the front of the first case for allowing touch access to controls on the machine pump;
a belt loop attached to the back of the first case;
a first portal on the first case for accepting tubes for connecting to the machine pump;
providing a second case having an opening, the second case configured to accept the feeding bag, the second case further comprised of;
a top flap for covering the opening in the second case;
a belt loop attached to the back of the second case;
a portal on the bottom of the second case for accepting a tube for connecting to the feeding bag;
placing the feeding bag into the second case, the feeding bag having a food supply tube with a first and second end, the first end connected to the bottom of the feeding bag;
placing the second end of the food supply tube through the portal at the bottom of the second case;
placing the second end of the food supply tube through the first portal on the first case;
connecting the second end of the food supply tube to the machine pump;
providing a delivery tube for delivering food to the user, the delivery tube having a first and second end;
connecting a first end of the delivery tube to the machine pump;
placing the delivery tube through the first or second portal on the first case;
placing the machine pump into the first case with the control panel viewable through the viewing window;
closing the top flap of the first case;
placing a belt through the belt loops on the first and second cases;
placing the belt around the user's waist;
attaching the second end of the delivery tube to the user; and
starting the machine pump.

20. A method according to claim 19, wherein the steps recited in claim 19 are performed in order as listed.

21. A method according to claim 19, further comprising the step of:
moving the first and second cases on the belt to positions comfortable to the user.

22. A method according to claim 19, further comprising the step of:
replacing the feeding bag from the second case without removing the cases from the user's waist.

23. A method according to claim 19, wherein the first case is further comprised of a front flap for covering the viewing window, the method further comprising the steps of:
opening the front flap; and
controlling the machine pump through the window on the first case.
